(19) United States
(12) Patent Application Publication
(10) Pub. No.: US 2024/0269668 A1
(43) Pub. Date: Aug. 15, 2024

(54) Systems and Methods for Assessment of Lipid Membrane-Associated Molecule Kinetics, Dynamics, and Interactions

(71) Applicant: California Institute of Technology, Pasadena, CA (US)

(72) Inventor: Vishal Maingi, Altadena, CA (US)

(73) Assignee: California Institute of Technology, Pasadena, CA (US)

(21) Appl. No.: 18/486,054

(22) Filed: Oct. 12, 2023

Related U.S. Application Data
(60) Provisional application No. 63/415,546, filed on Oct. 12, 2022.

(51) Int. Cl. B01L 3/00; G01N 33/68; G01N 33/92

(52) U.S. Cl. CPC B01L 3/502; G01N 33/68; G01N 33/92; B01L 2200/02; B01L 2300/0832; G01N 2333/705

(57) ABSTRACT

Systems and methods for assessment of lipid membrane-associated molecules are described. Generally, a system can comprise a scaffold with a molecule attached thereon. The scaffold can comprise a plurality of hydrophobic seeds to form a liposome. The molecule attached to the scaffold can integrate into the formed liposome and then be released from the scaffold. The kinetics, dynamics, and interactions of the molecule within the liposome can be detected or monitored.

### CROSS REFERENCE TO RELATED APPLICATIONS

This application claims priority to U.S. Provisional Application Ser. No. 63/415,546 entitled “On-Chip Synthesis and Purification of Digital Nanoreactors to Assay Membrane Protein Interactions,” filed Oct. 12, 2022, which is incorporated herein by reference in its entirety.

### STATEMENT OF FEDERALLY SPONSORED RESEARCH

This invention was made with government support under Grant No. MH125320 awarded by the National Institutes of Health. The government has certain rights in the invention.

### TECHNICAL FIELD

The disclosure is generally directed to systems and methods to assess kinetics, dynamics and/or interactions of lipid membrane-associated molecules.

### BACKGROUND

Biological cells can trigger changes in their activity and protein expression via signaling. Generally, an external ligand compound can come in contact with one or more proteins with the cell membrane to generate a signaling cascade. The membrane proteins can provide signals to intracellular proteins for downstream activity important for several cellular functions, including cell survival and metabolism. How membrane proteins interact could reveal many important insights about cellular function at the molecular level. Characterizing membrane protein interactions is important to understand their function and their mechanistic role in pathogenesis, and also useful for the drug discovery process. A report in 2006 suggested that more than 60% of the targets of medicinal compounds are membrane proteins (J. P. Overington, et al., Nat. Rev. Drug Discov. 2006, 5, 993-996, the disclosure of which is incorporated herein by reference). However, for many membrane proteins it is difficult to determine whether their active state is in the form of monomer or oligomer (interaction between two or more membrane proteins). Experimental methods developed so far cannot control how the absolute number of proteins could be programmed to interact in an isolated native lipid environment. Thus, the oligomeric functional states for many membrane proteins remain elusive or controversial.

Experimental analytical approaches used so far to identify the functional oligomeric states each have limitations and often it is necessary to employ multiple approaches to determine the oligomeric state of the proteins. For example, polyacrylamide gels cannot provide a native lipid environment in which membrane proteins function; bulk molecular Förster resonance energy transfer (FRET) methods have issues such as inter-oligomeric FRET and are concentration sensitive whereas single molecular FRET methods employed to resolve oligomeric states are practically challenging; mass spectrometry requires expensive instrumentation and samples are sometimes prepared with detergents; chemical cross-linking can be done to form stable dimers but it can be unspecific leading to formation of undesired dimers or oligomers; and digital nanoreactors designed for stoichiometric and spatiotemporal control of receptors is laborious, requires long purification steps, difficult to adapt for faster screening, and only suitable for chemical induced interactions (see, V. Maingi, et al., Nat Commun. 2023, 14(1):1532, the disclosure of which is incorporated herein by reference).

To avoid the drawbacks of the common methods, for example described above, an innovative platform is presented here to enable membrane protein interactions (or any molecular receptors; synthetic or natural or chimeric proteins etc.) in a cell-free but native lipid environment and to measure real time interactions, kinetics, and dynamics. In contrast to digital nanoreactors, the platform disclosed here has following important features: a) absolute stoichiometry, spatial and temporal (SST) control over the molecular receptor interactions without any cross talk between the platforms, and b) the surface-based version of these platforms would allow faster purification protocol. Importantly, both these features could make it suitable for developing methods to screen hundreds of membrane proteins with SST control. Here, absolute stoichiometry means exact numbers of molecular receptors (same or different) in any ratio or combination, spatial control means the molecular receptors could be initially positioned/tethered at desirable locations well-separated apart, and temporal control means molecular receptors can be released to interact when desired.

### SUMMARY

Several embodiments are directed towards systems and methods to assess behavior of lipid membrane-associated molecule. A system can comprise a scaffold having a tubular shape with one or more molecules attached to the scaffold within the lumen. The scaffold can further comprise a means for forming liposome within the lumen. The one or more molecules can integrate or otherwise associate with the liposome. The one or more molecules can be released from the scaffold. The behavior of the released molecules within the liposome can be detected and/or monitored.

In some implementations, a system is for assessing lipid membrane-associated molecules.

In some implementations, the system comprises a molecular scaffold having a tubular shape with open terminal ends.

In some implementations, the system comprises one or more molecules, each molecule of the one or more molecules reversibly tethered to the molecular scaffold within a lumen of the molecular scaffold.

In some implementations, the system comprises a plurality of hydrophobic seeds tethered to the molecular scaffold within a lumen of the molecular scaffold.

In some implementations, the system comprises two or more molecular scaffolds, wherein each molecular scaffold has a tubular shape and is conjoined with another molecular scaffold in an end-to-end manner such that the lumen of each molecular scaffold is conjoined.

In some implementations, each molecular scaffold comprises one or more molecules reversibly tethered thereto within its lumen.

In some implementations, each molecular scaffold is conjoined with another molecular in an end-to-end manner such that gaps between the conjoined molecular scaffolds are mitigated or absent.

In some implementations, each molecular scaffold is conjoined with another molecular via complementary oligomeric nucleic acids.

In some implementations, each molecular scaffold is conjoined with another molecular such that the plurality of hydrophobic seeds tethered to the molecular scaffold are patterned in a manner such that it can allow a molecule to pass thereby via a lipid membrane of a liposome formed by the plurality of hydrophobic seeds.

In some implementations, the molecular scaffold is configured and the one or more molecules are tethered to the scaffold in a manner such that the one or more molecules are sterically hindered from interacting with a molecule tethered within a lumen another molecular scaffold.

In some implementations, each molecule of the one or more molecules is capable of associating with a lipid membrane.

In some implementations, at least one molecule of the one or more molecules comprises a hydrophobic domain.

In some implementations, at least one molecule of the one or more molecules comprises an attached hydrophobic moiety.

In some implementations, at least one molecule of the one or more molecules comprises a transmembrane protein, a peripheral membrane protein, a nucleic acid with attached hydrophobic domain, a lipid, a fatty acid, a phospholipid, a glycolipid, a sphingolipid, a glycerolipid, a sterol, cholesterol, a prenol, a saccharolipid, a tocopherol, a hydrocarbon chain, and a polyketide.

In some implementations, the reversible tether a nucleic acid oligomer.

In some implementations, strand displacement is capable of untethering the molecule.

In some implementations, the reversible tether comprises a labile chemical structure or functional group.

In some implementations, the tether is capable of being cleaved via energy to break the labile chemical structure or functional group.

In some implementations, the plurality of hydrophobic seeds tethered to the molecular scaffold are patterned in a manner such that it can allow a molecule to pass thereby via a lipid membrane of a liposome formed by the plurality of hydrophobic seeds.

In some implementations, the system comprises a liposome formed within the lumen of the scaffold.

In some implementations, the liposome is formed via the plurality of hydrophobic seeds and lipid membrane components.

In some implementations, the one or more molecules are integrated within a lipid membrane of the liposome.

In some implementations, a method for assessing lipid membrane-associated molecules.

In some implementations, the method comprises providing a scaffold system in a solution, the scaffold system comprises: a molecular scaffold having a tubular shape, one or more molecules, each molecule of the one or more molecules reversibly tethered to the molecular scaffold within a lumen of the molecular scaffold, and a plurality of hydrophobic seeds tethered to the molecular scaffold within a lumen of the molecular scaffold.

In some implementations, the method comprises adding lipid membrane components to the solution such that a liposome is formed within the lumen of the molecular scaffold via the plurality of hydrophobic seeds.

In some implementations, the one or more molecules integrates within a lipid membrane of the liposome.

In some implementations, the method comprises untethering the one or more molecules from the scaffold.

In some implementations, the method comprises detecting a behavior of the one or more molecules within lipid membrane of the liposome.

In some implementations, the method comprises synthesizing the scaffold system.

In some implementations, the synthesizing the scaffold system comprises providing the molecular scaffold, wherein the molecular scaffold comprises: a tether for each molecule of the one or more molecules, and a tether for each hydrophobic seed of the plurality of hydrophobic seeds.

In some implementations, the synthesizing the scaffold system comprises attaching each molecule of the one or more molecules to a tether.

In some implementations, the synthesizing the scaffold system comprises attaching each hydrophobic seed to a tether.

In some implementations, the scaffold system comprises two or more molecular scaffolds, each molecular scaffold comprising a set of connectors to connect with another molecular scaffold.

In some implementations, the synthesizing the scaffold system comprises conjoining, using the set of connectors, each molecular scaffold to another molecular scaffold in an end-to-end manner such that the lumen of each molecular scaffold is conjoined.

In some implementations, the connectors comprise complimentary nucleic acid oligomers.

In some implementations, each molecular scaffold comprises one or more molecules reversibly tethered thereto within its lumen.

In some implementations, each molecular scaffold is conjoined with another molecular in an end-to-end manner such that gaps between the conjoined molecular scaffolds are mitigated or absent.

In some implementations, the method comprises attaching the molecular scaffold to a substrate via a linker, wherein the linker extends from an outer wall or inner wall or any component of the molecular scaffold to the substrate.

In some implementations, the method comprises removing an excess of molecular scaffolds, an excess of molecules, or an excess of lipid components from the solution by removing overlaid solution while the molecular scaffold remains attached to the substrate.

In some implementations, the step of adding to the solution lipid membrane components is done in presence of a detergent.

In some implementations, the method comprises exchanging a buffer via dialysis to remove the detergent from the solution.

In some implementations, the step untethering the one or more molecules from the scaffold comprises strand displacement, wherein the one or more molecules are reversibly tethered to the molecular scaffold via complimentary nucleic acid oligomers.

In some implementations, the step untethering the one or more molecules from the scaffold comprises impinging energy on a labile chemical structure of a tether that is utilized to tether the one or more molecules to the molecular scaffold.

In some implementations, at least one molecule comprises a molecular tag.

In some implementations, the step of detecting a behavior of the one or more molecules comprises detecting the molecular tag.

In some implementations, the one or more molecules comprises one or more fluorophores, a donor-acceptor fluorophore pair, or a quencher.

In some implementations, the step of detecting a behavior of the one or more molecules comprises monitoring kinetics, dynamics, or interactions of the one or more fluorophores.

In some implementations, the step of detecting a behavior of the one or more molecules comprises monitoring kinetics, dynamics, or interactions of the donor-acceptor fluorophore pair.

In some implementations, the step of detecting a behavior of the one or more molecules comprises monitoring kinetics, dynamics, or interactions via quenching of the fluorophore by the quencher.

In some implementations, the method comprises adding a stimulus to the scaffold system in a solution prior to the step of detecting a behavior of the one or more molecules

In some implementations, the molecular scaffold is configured and the one or more molecules are tethered to the scaffold in a manner such that the one or more molecules are sterically hindered from interacting with a molecule tethered within a lumen of another molecular scaffold.

### BRIEF DESCRIPTION OF THE DRAWINGS

The description and claims will be more fully understood with reference to the following figures, which are presented as exemplary embodiments of the disclosure and should not be construed as a complete recitation of the scope of the disclosure.

FIGS. 1A and 1B provide an example of a tubular molecular scaffold for use in a scaffolded liposome system. FIG. 1A provides a side view of the molecular scaffold. FIG. 1B provides a luminal view of the molecular scaffold.

FIGS. 2A to 2D provide examples of tubular molecular scaffolds for use in a scaffolded liposome system that comprises two or more molecular scaffolds. FIG. 2A provides a side view of a first molecular scaffold. FIG. 2B provides a luminal view of the first molecular scaffold. FIG. 2C provides a side view of the first molecular scaffold and a second molecular scaffold and the conjoining of thereof. FIG. 2D provides a side view of three molecular scaffolds conjoined together.

FIGS. 3A to 3D provide an example of synthesizing and using a scaffolded liposome system.

FIGS. 4A to 4F provide an example of synthesizing and using a scaffolded liposome system that comprises two molecular scaffolds.

### DETAILED DESCRIPTION

Turning now to the drawings and data, systems and methods for assessment of lipid membrane-associated molecules can comprise a scaffolded liposome system. The scaffolded liposome system can be utilized as a surrogate for assessing the behavior, kinetics, dynamics, and interactions of one or more lipid membrane-associated molecules within a cellular membrane. In several embodiments, the scaffolded liposome system comprises a scaffold, a means for forming a liposome, and means for tethering and releasing one or more lipid membrane-associated molecules within the liposome for assessment. In many embodiments, the scaffold of the scaffolded liposome system comprises a means for preventing premature interaction of the tethered molecules (e.g., during the assembly of the liposomes inside the lumen of scaffolds tethered with the molecules) and lipid membrane-associated molecules (e.g. to prevent cross-talk between the assembled scaffolded liposome systems with tethered or released molecules). In several embodiments, the scaffolded liposome system comprises a set of one or more scaffolds with one or more molecules capable of associating with a hydrophobic membrane tethered within the set of scaffolds. In many embodiments, the scaffolded liposome system comprises a set of one or more hydrophobic seeds and a set of lipids (and/or other hydrophobic molecules) for forming a liposome within the scaffold. Upon formation of a liposome, in several embodiments, the one or more molecules capable of associating with a hydrophobic membrane integrate within the liposome and then are released from their tether to assess the kinetics, dynamics, and/or interactions of the molecule within the liposome.

It is a goal of the current disclosure to describe a system for assessment of lipid membrane-associated molecule kinetics, dynamics, and/or interactions. Experimental analytical approaches used so far to assess membrane interactions each have limitations. Often, comprehensive assessment of lipid membrane-associated molecule interactions requires employment of multiple approaches. Several techniques have focused on interaction of membrane protein interactions. For example, polyacrylamide gels are utilized to assess proteins, but cannot provide a native lipid environment in which membrane proteins function. Bulk molecular Förster resonance energy transfer (FRET) methods are common to assess interaction of membrane proteins but have issues such as inter-oligomeric FRET and are concentration sensitive and single molecular FRET methods employed to resolve oligomeric states are challenging to implement. Assessment of membrane proteins via mass spectrometry requires expensive instrumentation and samples are often prepared with detergents, rendering it difficult to assess native interactions. Chemical cross-linking can help form stable dimers to improve interaction assessment, but can result in formation of unspecific interactions leading to inaccurate conclusions. Digital nanoreactors are a new technology that have been designed for assessment of membrane receptors with stoichiometric and spatiotemporal control, but the methodology is laborious, requires long purification steps, is difficult to adapt for faster screening, and is not suitable for interactions that occur without forced inducement (see, V. Maingi, et al., Nat Commun. 2023, 14(1):1532, the disclosure of which is incorporated herein by reference).

To avoid the drawbacks of prior methods, such as in the examples described above, a system is presented here to enable assessment of lipid membrane-associated molecule interactions (e.g., interactions of any molecular receptors, whether synthetic, natural, chimeric, or otherwise modified and/or combined). The system can be used in a cell-free environment and can measure real time interaction kinetics and dynamics of lipid membrane-associated molecule interactions. The current system disclosed here has so important features, such as absolute stoichiometry, spatial and temporal (SST) control over the lipid membrane-associated molecule interactions without any cross talk individualized systems, and can yield faster protocols of assessment when the systems are bound to a surface (facilitating quicker purification without the need of separation techniques such as centrifugation). The systems and methods described herein can be used to screen hundreds of membrane proteins with SST control. Here, absolute stoichiometry means exact numbers of lipid membrane-associated molecules (same or different) in any ratio or combination, spatial control means the lipid membrane-associated molecules can be initially positioned/tethered at a particular location in accordance with configured scaffolds, and temporal control means lipid membrane-associated molecules can controllably be released from a tether at a particular time to assess their interactions.

Several embodiments are directed to a scaffolded liposome system for assessment of lipid membrane-associated molecule kinetics, dynamics, and/or interaction. In many embodiments, a scaffolded liposome system comprises a molecular scaffold with a plurality of reactive groups for attachment of molecules, such that it can act like a pegboard. In several embodiments, a scaffold is formed in a tubular shape having an inner lumen. The scaffold can have a length extend along an axis with open terminal ends and can be any tubular shape, such that the scaffold walls form a circular, ovular, polygonal tube. Further, the lumen diameter can be constant the length of the scaffold or can vary (e.g., conical shaped ends). In some embodiments, a scaffold is formed using DNA origami, but any material having a plurality of available reactive groups such that tethers and/or molecules can be appended thereon can be utilized. For more on DNA origami scaffolds and methods of synthesis, see, e.g., S. F. J. Wickham, et al., Nat Commun. 2020, 11(1):5768, the disclosure of which is incorporated herein by reference.

In many embodiments, a scaffold further comprises a set of one or more tethers facing towards a lumen. In several embodiments, each tether is configured to connect with a molecule capable of associating with a lipid membrane. Any molecule (especially molecule) capable of being utilized as a tether for connecting with a molecule capable of associating with a lipid membrane. In some embodiments, the tether is a single-stranded nucleic acid oligomer (or aptamer), which can connect with a molecule bound to a complementary single-stranded nucleic acid oligomer (or aptamer). In some embodiments, a nucleic acid oligomer (or aptamer) for use as a tether is between 10 and 200 base pairs, but can be adjusted based on complementation and reaction conditions.

In many embodiments, the scaffold and tether are configured such that when the molecule is in connection with the tether (i.e., the molecule is tethered to the scaffold), the molecule is incapable of interacting with other molecules present in a lumen of another scaffold and any other molecules tethered in the same lumen. In several embodiments, the tether is configured such that when the molecule is in connection with the tether (i.e., the molecule is tethered to the scaffold), the molecule is capable of integrating within a lipid bilayer of a liposome formed inside the lumen of the scaffold. In some embodiments, the tether is configured to be able to release the molecule capable of associating with a lipid membrane, which can be done by any appropriate chemistry.

In several embodiments, a scaffolded liposome system comprises one or more molecules capable of associating with a hydrophobic membrane. In many embodiments, the one or more molecules are reversibly tethered to the scaffold. Any mechanism for reversible tethering can be utilized. In some implementations, the tether is a nucleic acid oligomer (or aptamer) and complement, with one strand attached to the scaffold and the complement strand attached to the molecule; in these embodiments, strand displacement can be used to release the molecule from the scaffold (see, e.g., F. C. Simmel, et al., Chem Rev. 2019, 119(10):6326-6369, the disclosure of which is incorporated herein by reference). In some implementations, the tether comprises a labile chemical structure capable of being cleaved via energy to hydrolyze or otherwise break the labile chemical structure and release the molecule (e.g., photolabile structure and light energy; e.g., redox-labile structure and redox energy; e.g., heat-labile structure and heat).

In many embodiments, when a liposome is formed within the scaffold, the molecule integrates within the lipid bilayer of the liposome while remaining tethered to the scaffold. In several embodiments, the molecule is released from the tether to assess the kinetics, dynamics, and/or interactions of the molecule associated with the lipid bilayer of the liposome. In some implementations, two or more unique or same lipid membrane-associated molecules are tethered to a scaffolded liposome system, in which the molecules capable to integrate with the lipid bilayer of the liposome are then released from their tether to determine their kinetics, dynamics, and interaction.

Any lipid membrane-associated molecule capable of integrating or otherwise associating with a lipid membrane can be utilized. In several embodiments, the lipid membrane-associated molecule comprises a hydrophobic domain or an attached hydrophobic moiety capable of integrating and/or associating with a lipid membrane. Examples of lipid membrane-associated molecules include (but are not limited to) transmembrane proteins, peripheral membrane proteins, nucleic acids with attached hydrophobic domain, hydrocarbon molecules, lipids, fatty acids, phospholipids, glycolipids, sphingolipids, glycerolipids (e.g., mono-, di-, tri-glycerol), sterols, cholesterol, prenols, tocopherols, saccharolipids, and polyketides. Any molecule (especially peripheral membrane proteins) can be lipid anchored by incorporating a covalently attached fatty acid acyl chain via palmitoylation, myristoylation, or prenylation. The lipid membrane-associated molecule can be natural or synthetic. Several protocols allow for cell-based expression (mammalian, bacterial, insect, plants etc.) or cell-free synthesis of lipid membrane-associated proteins and molecules (see, e.g., Z. A. Manzer, et al., Trends Biochem Sci. 2023, 48(7):642-654; e.g., H. Moghimianavval, et al., Methods Mol Biol. 2022; 2433:105-120; the disclosures of which are each incorporated herein by reference).

In several embodiments, a lipid membrane-associated molecule comprises a molecular tag. A molecular tag is a chemical moiety that enables detection and/or monitoring of molecules within the system (e.g., tethered to scaffold or within the lipid bilayer of a liposome). Molecular tags can be expressed as part of the molecule and/or chemical moieties that are attached to the molecule. Molecular tags include (but are not limited to) protein expression tags (e.g., HA-tag, FLAG-tag), DNAzymes (including split DNAzymes), enzymes (including split enzymes) that can generate an observable product (e.g., horseradish peroxidase, luciferase), conjugated nucleic acid oligomers (which can initiate DNA, RNA and/or enzyme based cascade reactions to produce any direct or indirect observables), fluorophores (e.g., fluorescent proteins, split fluorescent proteins, fluorescent dyes), quenchers, antibodies, metals (e.g., gold), donor-acceptor fluorophore pairs, and fluorophore-quencher pairs. In some embodiments, two or more lipid membrane-associated molecule can each comprise a tag system, such as (for example) a donor-acceptor fluorophore pair, split-DNAzymes, split enzymes, split fluorescent proteins, and a fluorophore-quencher pair. In some implementations, a first lipid membrane-associated molecule comprises a donor and a second lipid membrane-associated molecule comprises an acceptor to yield a donor-acceptor fluorophore pair that emits a particular fluorescence when in proximity. In some implementations, a first lipid membrane-associated molecule comprises a first fragment of a split DNAzyme and a second lipid membrane-associated molecule comprises a second fragment of a split DNAzyme to yield a complete DNAzyme when in proximity such that the DNAzyme is in an activated form. In some implementations, a first lipid membrane-associated molecule comprises a first fragment of a split enzyme and a second lipid membrane-associated molecule comprises a second fragment of a split enzyme to yield a complete enzyme when in proximity such that the enzyme is in an activated form. In some implementations, a first lipid membrane-associated molecule comprises a first fragment of a split fluorescent protein and a second lipid membrane-associated molecule comprises a second fragment of a split fluorescent protein to yield a complete fluorescent protein when in proximity such that the fluorescent protein is in a form that can emit a fluorescence. In some implementations, a first lipid membrane-associated molecule comprises a fluorophore and a second lipid membrane-associated molecule comprises a quencher to yield a fluorophore-quencher pair quench that quenches the fluorescent signal when in proximity. When assessing fluorescence, any suitable detector, camera, or plate reader instrument can be utilized. In some implementations, single-molecule microscopy or total internal reflection fluorescence (TIRF) detection is utilized. In some embodiments, an optical system is utilized to impinge light energy onto a fluorophore.

To assess an interaction between lipid membrane-associated molecules, a form of stimulation may be required. Accordingly, in some embodiments, a scaffolded liposome system comprises a stimulus to induce an interaction between lipid membrane-associated molecules. Examples of stimuli include (but are not limited to) a ligand, drug compound, light energy, pH, ionic condition, temperature, and post-translational modification (e.g., phosphorylation).

In many embodiments, a scaffolded liposome system comprises a set of one or more scaffolds. In embodiments in which a system utilizes a single scaffold, the scaffold can comprise a set of one or more hydrophobic seeds and a set of one or more tethers, where each tether is individually in connection with a molecule capable of associating with a lipid membrane. When a single scaffold is utilized, in some embodiments, one or more molecules are each individually attached to the scaffold. In some embodiments, the two- or more molecules are not capable of interacting with the other during setup of the scaffolded liposome system; either they require a stimulus to interact or require some additional molecules/conditions (ligands, ions, detergents, pH, temperature) to inhibit their interaction initially before they get tethered at their respective tethering sites inside the lumen. In some implementations, the two or more unique molecules require a stimulus to interact. In some implementations, at least one molecule of the two or more molecules comprises a molecular appendage (e.g., a cage molecule) that prevents interaction between the two molecules, where the molecular appendage can be released after tethering the two or more molecules. Any mechanism for caging and releasing a cage molecule can be utilized.

In several embodiments, a scaffolded liposome system comprises a set of two or more scaffolds, where each scaffold of the set is configured to connect with at least one other scaffold. In embodiments in which a system utilizes two or more scaffolds, each scaffold can comprise a set of one or more tethers, each set of tethers in connection with a molecule capable of associating with a lipid membrane. When two or more scaffolds are utilized, in some embodiments, one or more molecules are each individually attached to one of the two or more scaffolds. By utilizing two or more scaffolds that interconnect, a unique molecule can be tethered to its own scaffold in its own unique reaction without coming in contact with another molecule. This allows for two or more unique scaffolds to be synthesized with unique molecules tethered that can be later connected to form an interconnected set of two or more scaffolds to yield the scaffolded liposome system. Accordingly, two or more molecules can be assessed for their interaction via the scaffolded liposome system without premature interactions during synthesis of the scaffolded liposome system.

In some embodiments of a scaffolded liposome system that comprises a set of two or more scaffolds, a first scaffold comprises a set of one or more connectors configured to interconnect with a set of one or more connectors of a second scaffold such that the first scaffold and the second scaffold conjoin via the connectors. Any molecule (especially molecule) or a chemical linker capable of being utilized as a connector for connecting two scaffolds can be utilized. In some embodiments, the scaffold connector is a single-stranded nucleic acid oligomer (or aptamer) bound to a first scaffold, which can connect with a complementary single-stranded nucleic acid oligomer (or aptamer) bound to a second scaffold or connect directly to the scaffold (e.g. for DNA origami based scaffolded systems a single-stranded nucleic acid oligomer from the first scaffold hybridizing with the free long DNA scaffold of the second system). In some embodiments, a nucleic acid oligomer (or aptamer) for use as a scaffold connector is between 10 and 200 base pairs, but can be adjusted (>200 base pairs) based on complementation and reaction conditions. For more connectors to connect two or more scaffolds, see, e.g., S. F. J. Wickham, et al., Nat Commun. 2020, 11(1):5768; e.g., V. Maingi, et al., Nat Commun. 2023, 14(1):1532; the disclosures of which are each incorporated herein by reference.

In some embodiments, a set of one or more connectors of scaffold is at or near an end of the tubular scaffold such that the scaffold configured to connect with another scaffold in an end-to-end manner. In some implementations, when two scaffolds are conjoined end-to-end, the two scaffolds share a lumen. In some implementations, when two scaffolds are conjoined end-to-end, the walls of the two scaffolds are conjoined in a manner such that gaps therebetween are mitigated or absent.

In several embodiments, the scaffold further is configured to comprise a set of one or more hydrophobic seeds projected from the scaffold towards the lumen. The hydrophobic seeds can provide an initiator for forming a liposome. In many embodiments, the scaffold is configured such that a liposome can be formed therein via the set of hydrophobic seeds and addition of lipid membrane components and/or other hydrophobic molecules (or any hydrophobic molecule for forming a liposome). Any molecule (especially molecule or organic molecule) capable of being utilized as hydrophobic seed to initiate the formation of liposome can be utilized. In some embodiments, a hydrophobic seed comprises a hydrophobic molecule. Examples of hydrophobic molecules that can be utilized as hydrophobic seeds and/or lipid membrane components include (but are not limited to) peptides, peptoids, proteins, hydrocarbon molecules, lipids, fatty acids, phospholipids, glycolipids, sphingolipids, glycerolipids (e.g., mono-, di-, tri-glycerol), sterols, cholesterol, tocopherols, prenols, saccharolipids, and polyketides.

In several embodiments, the set of hydrophobic seeds project towards the scaffold lumen via a tether bound to the scaffold. Any molecule (especially molecule) capable of being utilized as a tether can be used for projecting hydrophobic seeds. In some embodiments, the tether is a single-stranded nucleic acid oligomer (or aptamer), which can connect with a molecule, connected to a hydrophobic seed, bound to a complementary single-stranded nucleic acid oligomer (or aptamer). In some embodiments, a nucleic acid oligomer (or aptamer) for use as a tether is between 10 and 200 base pairs, but can be adjusted (>200 base pairs) based on complementation and reaction conditions.

The tethers used for binding hydrophobic seeds may sterically hinder interaction between lipid membrane-associated molecules by blocking the ability of one or more of the lipid membrane-associated molecules from diffusing along the membrane. To counteract that potential issue, in some implementations, the tethers of the set of hydrophobic seeds are attached to the scaffold in a configuration to mitigate steric effects that can hinder lipid membrane-associated molecule interactions. Accordingly, in some implementations, the tethers of the set of hydrophobic seeds are attached to the scaffold in a configuration such that the ability of lipid membrane-associated molecules to traverse through the set of tethers is unhindered by the tethers or such that their hindrance is mitigated.

A scaffolded liposome system can be provided as free-floating systems in a solution or can be affixed to a substrate. By affixing a scaffolded liposome system, synthesis of the system can be greatly facilitated by removing the need for centrifugation (or another separation technique) during incubation and wash steps. Any suitable substrate material for attaching a linker can be utilized, such as (for example) glass, silica, SiₓN_{y}, poly-Lysine, poly-Ornithine, hyaluronic acid, polyacrylamide, sephadex, polystyrene, polytetrafluoroethylene, polyvinylchloride, and polycarbonate. To affix a scaffolded liposome system to a substrate, a linker can tether the outer scaffold wall with the substrate. Any molecule (especially molecule) capable of being utilized as a linker for tethering a scaffold to a substrate can be utilized. In some embodiments, the linker is a single-stranded nucleic acid oligomer (or aptamer) bound to the substrate, which can connect with a complementary single-stranded nucleic acid oligomer (or aptamer) bound to the scaffold. In some embodiments, a nucleic acid oligomer (or aptamer) for use as a substrate linker is between 10 and 200 base pairs, but can be adjusted (>200 base pairs) based on complementation and reaction conditions.

Several embodiments are directed to a system for assessing lipid membrane-associated molecule behavior. A system can comprise one or more of the various components described. In some embodiments, a system comprises one or more molecular scaffolds. In some embodiments, a system comprises one or more lipid membrane-associated molecules (or one or more molecules capable of associated with a membrane). In some embodiments, a system comprises one or more tethers for reversibly tethering a molecule to a scaffold. In some embodiments, a system comprises one or more hydrophobic seeds. In some embodiments, a system comprises one or more tethers for projecting a hydrophobic seed towards the scaffold lumen. In some embodiments, a system comprises a formed liposome. In some embodiments, a system comprises one or more connectors for connecting two scaffolds. In some embodiments, a system comprises a stimulus to induce an interaction between lipid membrane-associated molecules. In some embodiments, a system comprises a lipid membrane-associated molecule comprising a molecular tag. In some embodiments, a system comprises a detector, a camera, a microscope, or a plate reader instrument. In some embodiments, a system comprises an optical system for impinging light energy onto a fluorophore.

Several embodiments are directed to a kit for assessing lipid membrane-associated molecule behavior. A kit can comprise two or more of the various components described for assembling together a system. In some embodiments, a kit comprises one or more molecular scaffolds. In some embodiments, a kit comprises one or more lipid membrane-associated molecules (or one or more molecules capable of associated with a membrane). In some embodiments, a kit comprises one or more tethers for reversibly tethering a molecule to a scaffold and/or a substrate. In some embodiments, a kit comprises one or more hydrophobic seeds. In some embodiments, a kit comprises one or more tethers for projecting a hydrophobic seed towards the scaffold lumen. In some embodiments, a kit comprises a formed scaffolded liposome. In some embodiments, a kit comprises one or more connectors for connecting two scaffolds. In some embodiments, a system comprises a stimulus to induce an interaction between lipid membrane-associated molecules. In some embodiments, a kit comprises a lipid membrane-associated molecule comprising a molecular tag. In some embodiments, a kit is for use with a detector, a camera, or a plate reader instrument. In some embodiments, a kit is for use with an optical system for impinging light energy onto a fluorophore. In some embodiments, a kit is for to create a substrate with a tether for the surface attachment of the scaffolds or scaffolded liposome systems.

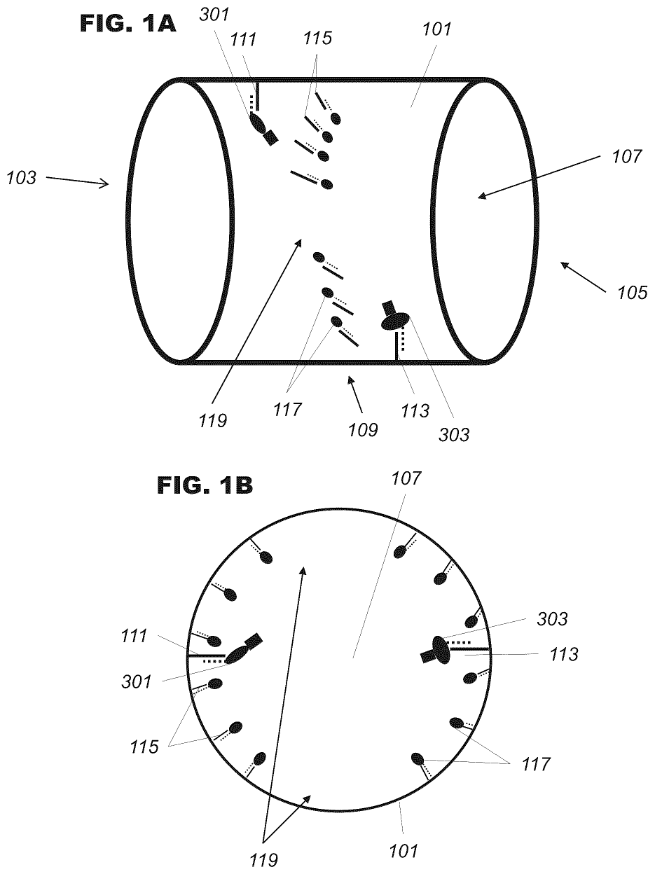

Provided in FIGS. 1A and 1B is an example of a molecular scaffold for use in a scaffolded liposome system to assess lipid membrane-associated molecule interactions. In this example, a molecular scaffold 101 has a tubular shape. Scaffold 101 includes a first end 103 and second end 105, each of which are open with a lumen 107 therebetween. As discussed herein, scaffold 101 can be any molecular scaffold having a plurality of available reactive groups such that tethers and/or molecules can be appended thereon, such as a DNA origami barrel.

Between a middle portion 109 and first end 103 is a first tether 111 and between the middle portion and second end 105 is a second tether 113. First tether 111 and second tether 113 each extend towards lumen 107 and is capable of attaching a molecule 301 or a molecule 303, which are capable associating with a lipid membrane. First tether 111 and second tether 113 are shown to be on opposite sides of lumen 107 and on opposite ends of scaffold 101, but their placement can be anywhere on the scaffold and can be dependent on assessment to be performed. In some implementations, the tubular length of the scaffold 101 and the placement of first tether 111 and second tether 113 is configured such that when a molecule is attached thereon, the scaffold walls sterically hinder the ability of the molecule to interact with molecules present in other similar scaffolded systems. Further, the placement and length of first tether 111 and second tether 113 should be configured such that an attached molecule is capable of integrating within a liposome formed within lumen 107. In some implementations, first tether 111 and second tether 113 are nucleic acid oligomers (or aptamers) configured to complement an oligomer with an attached molecule.

Within middle portion 109 of scaffold 101 are a plurality of tethers 115, each with an attached hydrophobic seed 117. In some implementations, each tether of the plurality of tethers 115 is nucleic acid oligomer (or aptamer) configured to complement a nucleic acid oligomer (or aptamer) with an attached hydrophobic seed 117. Each tether of the plurality of tethers 115 can project towards lumen 107 such that hydrophobic seed 117 is within the lumen. Hydrophobic seeds 117 can be used to initiate formation of a liposome when lipid membrane components are added to the scaffold system. Notably, the plurality of tethers 115 are in a configuration having a set of two extended gaps 119, which can allow for lipid membrane-associated molecules to pass therethrough when released to move freely about the liposome. Notably, any spacing of the tethers that allows for lipid membrane-associated molecules to pass therethrough can be utilized.

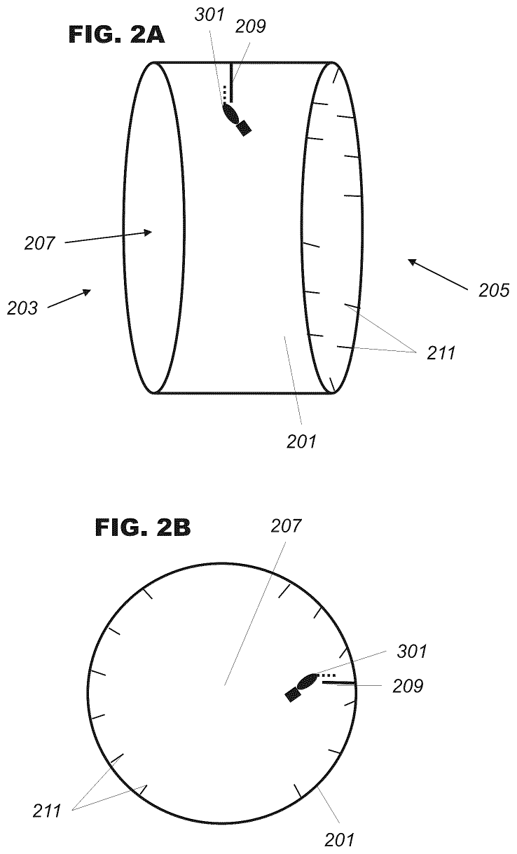

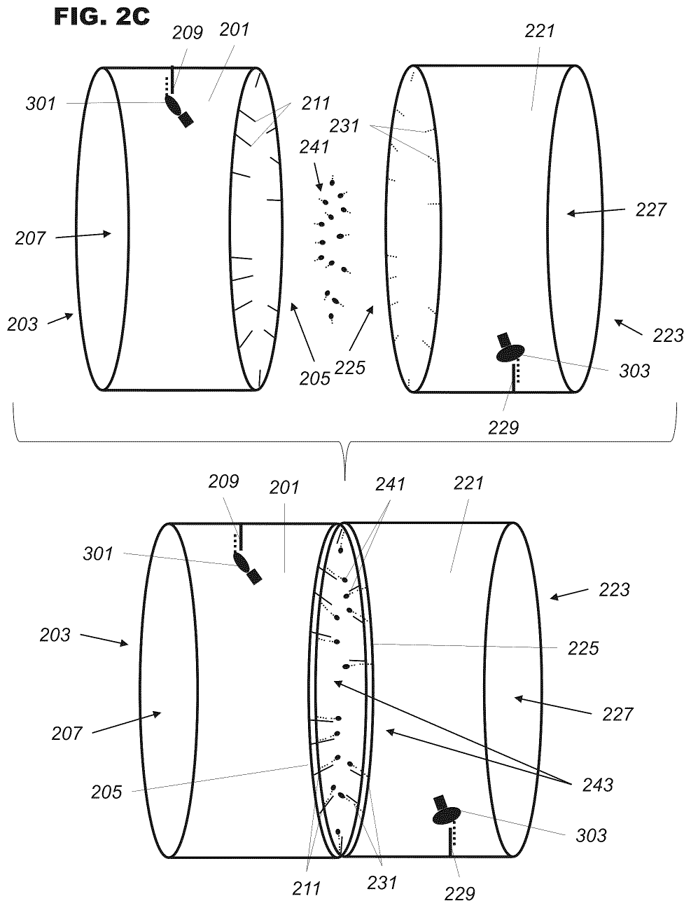

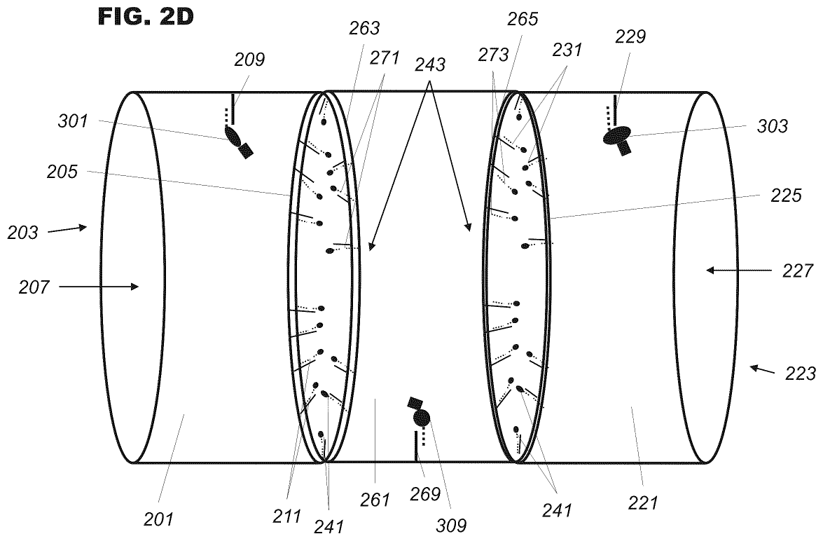

Provided in FIGS. 2A to 2D are examples of a molecular scaffold for use in a scaffolded liposome system comprising two or more scaffolds. FIGS. 2A and 2B provide an example of scaffold 201 configured to conjoin with a second scaffold. Scaffold 201 can comprise a tubular shape having a first end 203 and second end 205, each of which are open with a lumen 207 therebetween. Scaffold 201 can be any molecular scaffold having a plurality of available reactive groups such that tethers and/or molecules can be appended thereon, such as a DNA origami barrel.

Scaffold 201 includes a tether 209 between first end 203 and second end 205, both ends are open, with a lumen 207 therebetween, for connecting with a molecule 301, which is capable of associating with a lipid membrane. In some implementations, the tubular length of the scaffold 201 and the placement of tether 209 is configured such that when a molecule is attached thereon, the scaffold walls sterically hinder the ability of the molecule to interact with molecules present in other similar scaffolded systems. In some implementations, tether 209 is a nucleic acid oligomer (or aptamer) configured to complement an oligomer with an attached molecule.

Scaffold 201 further comprises a plurality of tethers 211 which are provided near second end 205. One or more of the plurality of tethers 211 is configured to be connectors for connecting with a second scaffold and one or more of the plurality of tethers 211 can provide a means for attaching a hydrophobic seed. In some implementations, each tether of the plurality of tethers 211 is nucleic acid oligomer (or aptamer) configured to complement a nucleic acid sequence or an oligomer (or aptamer) of a second scaffold as a connector or further configured to complement a nucleic acid oligomer (or aptamer) with an attached hydrophobic seed.

FIG. 2C provides an example of conjoining scaffold 201 with scaffold 221 and further attaching a plurality of hydrophobic seeds 241. Much like scaffold 201, scaffold 221 can comprise a tubular shape having a first end 223 and second end 225, with a lumen therebetween 227. Much like scaffold 201, scaffold 221 can be any molecular scaffold having a plurality of available reactive groups such that tethers and/or molecules can be appended thereon, such as a DNA origami barrel.

Scaffold 221 includes a tether 229 between first end 223 and second end 225 for connecting with a molecule 303 capable of associating with a lipid membrane. In some implementations, the placement of tether 229 is configured such that when a molecule is attached thereon, the scaffold walls sterically hinder the ability of the molecule to interact with molecules present in other similar scaffolded systems. In some implementations, tether 229 is a nucleic acid oligomer (or aptamer) configured to complement an oligomer with an attached molecule.

Scaffold 221 further comprises a plurality of tethers 231 which that are provided near second end 225. One or more of the plurality of tethers 231 is configured to be connectors for connecting with scaffold 201 via one or more of the plurality of tethers 211 or connecting directly to the scaffold 201. In some implementations, one or more tethers of the plurality of tethers 231 is nucleic acid oligomer (or aptamer) configured to complement a nucleic acid oligomer (or aptamer) one or more of the tether of plurality of tethers 211. Hydrophobic seeds 241 can be attached to a nucleic acid oligomer (or aptamer) configured to complement a nucleic acid oligomer (or aptamer) one or more of the tether of plurality of tethers 211 and or plurality of tethers 231.

Scaffold 201 and scaffold 221 are configured to conjoin together. One or more tethers of the plurality of tethers 211 is a complement of one or more of the tether of the plurality of tethers 231 or any direct component of the scaffold 221. This brings scaffold 201 to conjoin with scaffold 221. Specifically, second end 205 of scaffold 201 abuts second end 225 of scaffold 221 such that lumen 207 and lumen 227 join together to form one conjoined lumen. The 211 and/or plurality of tethers 231 can further be complementary with oligomers (or aptamers) attached to hydrophobic seeds 241, resulting in the hydrophobic seeds projecting towards the middle portion of the conjoined lumen. Hydrophobic seeds 241 can be used to initiate formation of a liposome when lipid membrane components are added to the molecular scaffold. Notably, plurality of tethers 211 and plurality of tethers 231 conjoin in a configuration having a set of two extended gaps 243, which can allow for lipid membrane-associated molecules to pass therethrough when released to move freely about the liposome. Notably, any spacing of the tethers that allows for lipid membrane-associated molecules to pass therethrough can be utilized.

FIG. 2D provides an example of scaffold system comprising scaffold 201, scaffold 221, and scaffold 261. Scaffold 201 and scaffold 221 are the same as described in reference to FIG. 2C except for the complementation of plurality of tethers 211 and plurality of tethers 231, which instead complement to plurality of tethers 271 and plurality of tethers 273, respectively. Scaffold 261 can comprise a tubular shape having a first end 263 and second end 265, each of which are open with a lumen therebetween. Scaffold 261 includes a tether 269 between first end 263 and second end 265 for connecting with a molecule 309, which is capable of associating with a lipid membrane. In some implementations, tether 269 is a nucleic acid oligomer (or aptamer) configured to complement an oligomer with an attached molecule.

Scaffold 261 further comprises a plurality of tethers 271 which that are provided near first end 263 and a plurality of 273 near second end 265. One or more of the plurality of tethers 271 is configured to be connectors for connecting with scaffold 201 via one of more of plurality of tethers 211 or any component of scaffold 201. Likewise, one or more of plurality of tethers 273 is configured to be connectors for connecting with scaffold 221 via one or more of plurality of tethers 231 or any component of scaffold 221. In some implementations, one or more tethers of the plurality of tethers 273 is nucleic acid oligomer (or aptamer) configured to complement one or more nucleic acid oligomer (or aptamer) of plurality of tethers 231 and one or more tethers of the plurality of tethers 273 is nucleic acid oligomer (or aptamer) further configured to complement a nucleic acid oligomer (or aptamer) with an attached hydrophobic seed. In some implementations, one or more tethers of the plurality of tethers 271 is nucleic acid oligomer (or aptamer) configured to complement one or more nucleic acid oligomer (or aptamer) tether of plurality of tethers 211. Hydrophobic seeds 241 can be attached to a nucleic acid oligomer (or aptamer) configured to complement one or more nucleic acid oligomer (or aptamer) tether of plurality of tethers 211, plurality of tethers 231, plurality of tethers 271, and/or plurality of tethers 273.

Although FIG. 2D depicts a scaffold system comprising three scaffolds joined end-to-end, any number of scaffolds can be conjoined end-to-end forming one long conjoined lumen. Accordingly, scaffolds and the molecules and/or hydrophobic seeds attached therein before or after conjoining the scaffolds can be configured in any number of ways and conjoined in any variety of combinations.

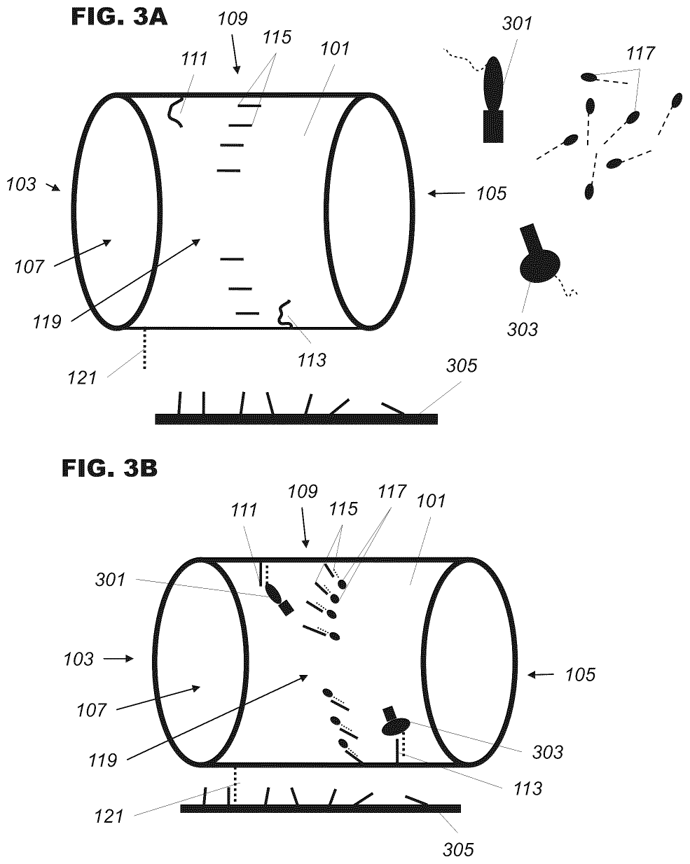

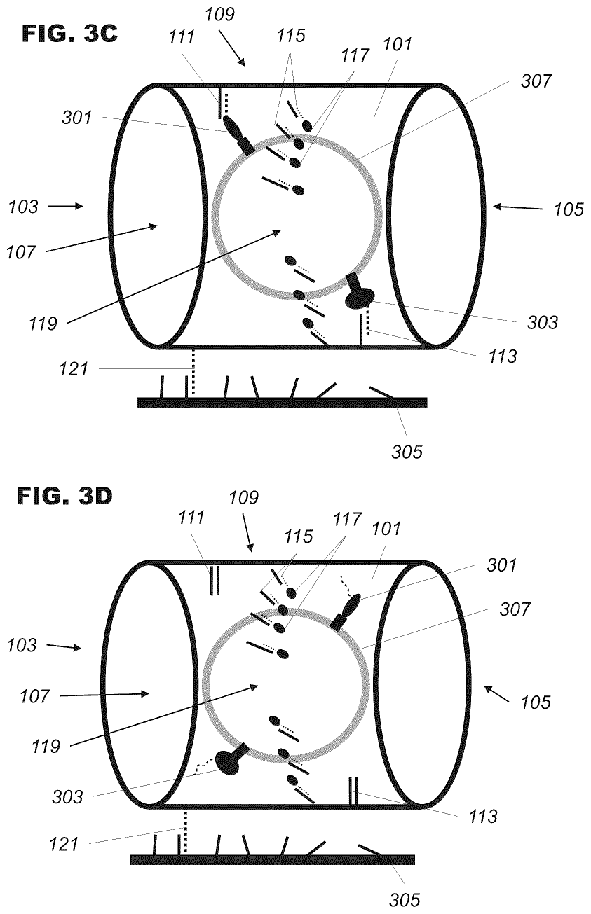

Provided in FIGS. 3A to 3D is an example of synthesizing and utilizing a scaffolded liposome system for assessment of kinetics, dynamics, and/or interaction of membrane-associate molecules. As shown in FIG. 3A, a scaffolded liposome system comprises a scaffold 101 as depicted in FIGS. 1A and 1B. Accordingly, scaffold 101 includes a first end 103 and second end 105, each of which are open with a lumen 107 therebetween. Scaffold 101 further comprises first tether 111 and second tether 113, each extending toward lumen 107. First tether 111 comprises a nucleic acid oligomer (or aptamers) configured to complement an oligomer (or aptamer) with attached molecule 301. Second tether 113 comprises a nucleic acid oligomer (or aptamers) configured to complement an oligomer (or aptamer) with attached molecule 303. There can be one or more of tethers similar to 111/113 and positioned anywhere within the lumen (of any length and diameter) depending on the requirements.

Within middle portion 109 of scaffold 101 are a plurality of tethers 115, where each tether of the plurality of tethers 115 is nucleic acid oligomer (or aptamer) configured to complement a nucleic acid oligomer (or aptamer) with an attached hydrophobic seed 117. Along the outer wall of scaffold 101 is tether 121 (one or more similar tethers), which is nucleic acid oligomer (or aptamer) configured to complement nucleic acid oligomer (or aptamer), one or more and forming any number of base pairs, extending from substrate 305. Substrate can be any suitable material for attaching linker thereto, as described herein.

To synthesize the scaffolded liposome system, tether 121 (one or more such tethers) is annealed/hybridized with an oligomer (or aptamer) attached to substrate 305 to link and affix scaffold 101 to the substrate. Annealing/hybridization can be performed with the substrate in solution with a suitable buffer at an appropriate temperature and reaction condition (e.g., 25° C. for 1 h). In some implementations, a plurality of scaffolds is linked and affixed to substrate, which can be done in a regular or irregular pattern. In some implementations, a plurality of scaffolds is linked and affixed to substrate using the same oligomer and complement as the linker. In some implementations, a plurality of scaffolds is linked and affixed to substrate, where each scaffold has a particular oligomer and complement such that each scaffold link and affix to the substrate in a particular pattern. Upon annealing, any unbound scaffolds can be removed and/or washed via changing of the overlaid solution. It should be noted, however, that in alternative implementations, scaffold 101 is not affixed to a substrate but instead is kept unaffixed in solution.

The scaffold system is further synthesized by annealing/hybridizing tether 111 with an oligomer (or aptamer) with attached molecule 301, annealing/hybridizing tether 113 with an oligomer (or aptamer) with attached molecule 303, and annealing/hybridizing the plurality of tethers 115 with a plurality of oligomers (or aptamers) with attached hydrophobic seed 117. These annealing/hybridization reactions can be performed concurrently or subsequently with the substrate in solution with a suitable buffer at an appropriate temperature and reaction condition (e.g., 25° C. for 1 h). Annealing/hybridization of molecules and/or oligomers with attached hydrophobic seed may be performed with detergent(s) to mitigate aggregation. Any unbound molecules and/or hydrophobic seeds can be removed and/or washed via changing of the overlaid solution either concurrently or in subsequent steps. If scaffold 101 is kept unaffixed in solution, centrifugation (e.g., ultracentrifugation) or alternative separation step can be performed to remove excess molecules and/or hydrophobic seeds either concurrently or in subsequent steps. The product of annealing/hybridization reactions is depicted in FIG. 3B.

With hydrophobic seeds 117 projecting towards lumen 107 upon completion of the annealing/hybridization reaction, a liposome can be formed. Lipid membrane components (e.g., but not limited to, any mixture of lipids, cholesterol) can be added to the solution. The lipid membrane components will associate with hydrophobic seeds 117 and form liposome 307 within lumen 107 (FIG. 3C). Liposome formation can be performed in solution at an appropriate temperature, suitable buffer with detergent, and reaction condition. In some implementations, detergent is slowly removed using buffer exchange technique (e.g., dialysis) to help formulate the liposomes. As liposome 307 forms, the hydrophobic portion of molecule 301 and of molecule 303 will integrate within the lipid bilayer of liposome while maintaining tethered to scaffold 101. Any free lipid membrane components remaining in solution can be removed and/or washed via changing of the overlaid solution. If scaffold 101 is kept unaffixed in solution, centrifugation (e.g., ultracentrifugation) or alternative separation step can be performed to remove excess lipid membrane components (see, e.g., Y. Yang, et al., Nat Chem. 2016, 8(5):476-483, the disclosure of which is incorporated herein by reference.

Upon liposome 307 formation and integration of molecule 301 and molecule 303 within the lipid bilayer of liposome, a reaction can be performed to release the molecules from their tether (FIG. 3D). In some implementations, strand displacement is performed to release molecule 301 and molecule 303 from scaffold 101. In some implementations, cleavage of a labile chemical structure is performed to release molecule 301 and molecule 303 from scaffold 101. With molecule 301 and molecule 303 released to move freely within lipid bilayer of the liposome, their kinetics, dynamics, and/or interaction can be assessed. In some implementations, a stimulus is added to the scaffolded liposome system to induce interaction between molecule 301 and molecule 303. In some implementations, scaffold 101 is detached from substrate 305 to perform the assessment, which can be performed by strand displacement or cleavage of a labile chemical structure of tether 121. In some implementations, molecule 301 and molecule 303 comprise a molecular tag to detect and/or monitor their behavior. In some implementations, molecule 301 and molecule 303 comprise one or more fluorophores, a donor-acceptor fluorophore pair, and/or a quencher to detect and/or monitor their behavior. In some implementations, a detector, a camera, or a plate reader instrument is utilized to detect and/or monitor the behavior of molecule 301 and molecule 303. In some implementations, single-molecule microscopy or total internal reflection fluorescence (TIRF) detection is utilized to detect and/or monitor the behavior of molecule 301 and molecule 303. In some implementations, an optical system is utilized to impinge light energy onto a fluorophore of molecule 301 and/or molecule 303.

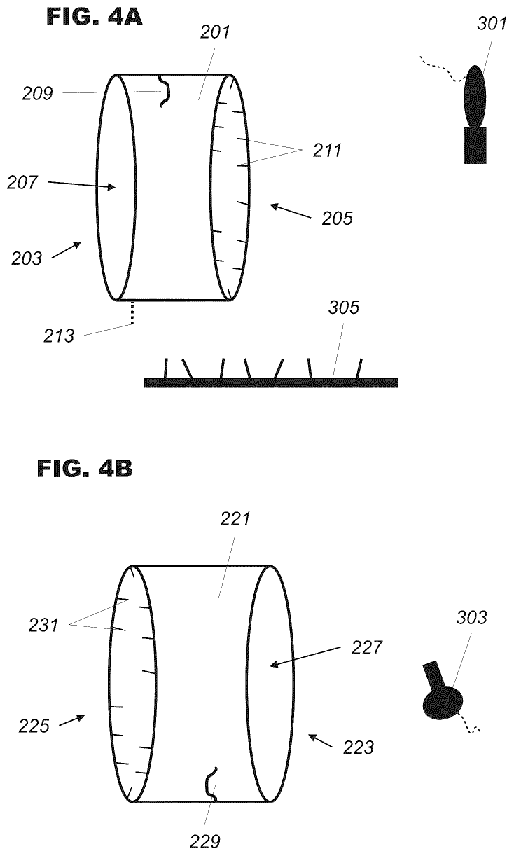

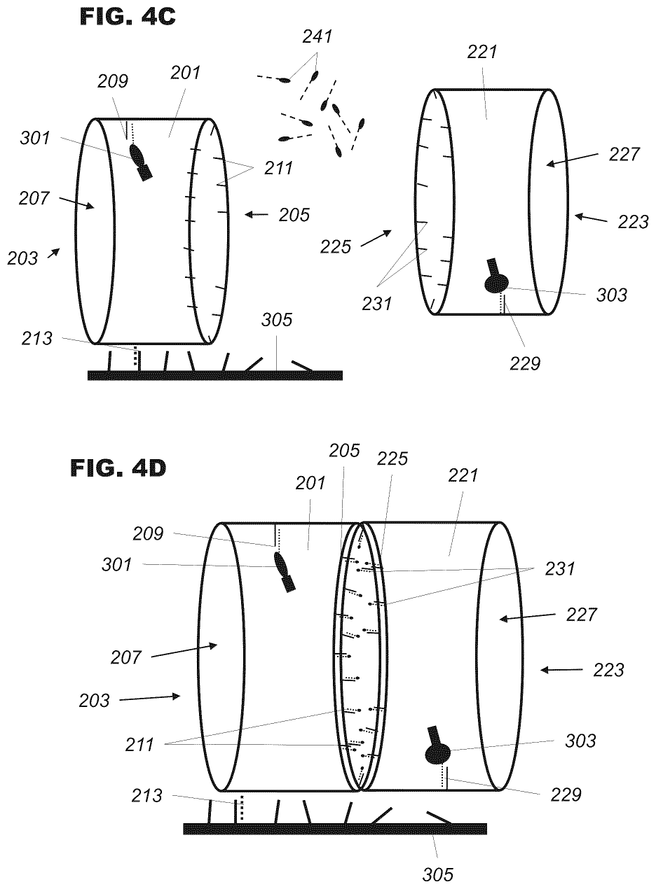

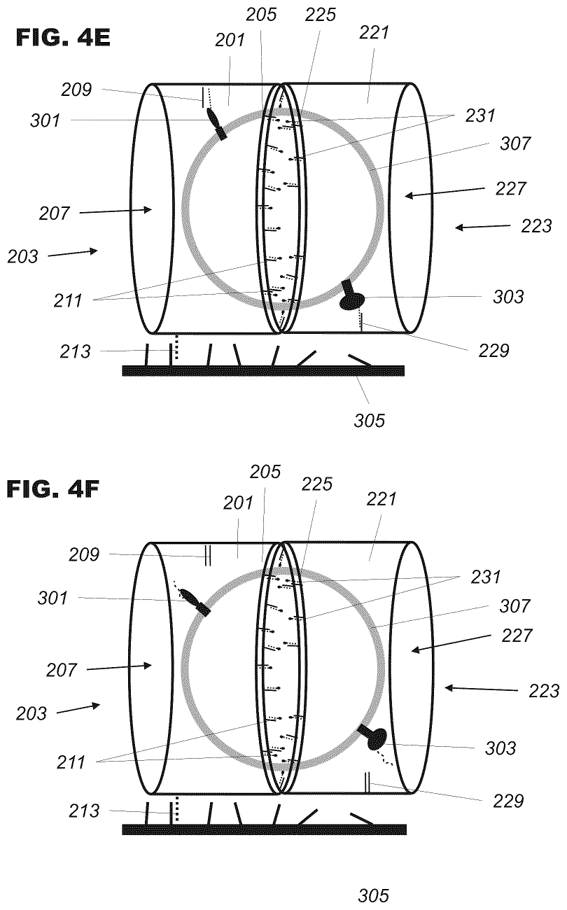

Provided in FIGS. 4A to 4F is an example of synthesizing and utilizing a scaffolded liposome system for assessment of kinetics, dynamics, and/or interaction of membrane-associate molecules. In this example, the scaffolded liposome system comprises two scaffolds that are conjoined similar to as depicted in FIG. 2C. As shown in FIG. 4A, a scaffolded liposome system comprises scaffold 201 as depicted in FIGS. 2A and 2B. Accordingly, scaffold 201 includes a first end 203 and second end 205, each of which are open with a lumen 207 therebetween. Scaffold 201 further comprises first tether 209 extending toward lumen 207. First tether 209 comprises a nucleic acid oligomer (or aptamers) configured to complement an oligomer (or aptamer) with attached molecule 301.

Near second end 205 of scaffold 201 are a plurality of tethers 211, where one or more tethers of the plurality of tethers 211 is nucleic acid oligomer (or aptamer) configured to be connectors and complement a nucleic acid oligomer (or aptamer) connector a second scaffold or connect directly to the second scaffold. One or more of the plurality of tethers 211 can further provide a means for attaching a hydrophobic seed. Along the outer wall of scaffold 201 is tether 213 (one or more such tethers), which is nucleic acid oligomer (or aptamer) configured to complement nucleic acid oligomer (or aptamer), one or more and forming any number of base pairs, extending from substrate 305. Substrate can be any suitable material for attaching linker thereto, as described herein.

To synthesize the scaffold system, tether 213 is annealed with an oligomer (or aptamer) attached to substrate 305 to link and affix scaffold 201 to the substrate. Annealing/hybridization can be performed with the substrate with a suitable buffer solution at an appropriate temperature and reaction condition (e.g., 25° ° C. for 1 h). In some implementations, a plurality of scaffolds is linked and affixed to substrate, which can be done in a regular or irregular pattern. In some implementations, a plurality of scaffolds is linked and affixed to substrate using the same oligomer and complement as the linker. In some implementations, a plurality of scaffolds is linked and affixed to substrate, where each scaffold has a particular oligomer (or aptamer) and complement such that each scaffold link and affix to the substrate in a particular pattern. Upon annealing/hybridization, any unbound scaffolds can be removed and/or washed via changing of the overlaid solution. It should be noted, however, that in alternative implementations, scaffold 201 is not affixed to a substrate but instead is kept unaffixed in solution.

The scaffolded liposome system is further synthesized by annealing/hybridizing tether 209 with an oligomer (or aptamer) with attached molecule 301. The annealing/hybridization reaction can be performed with a suitable buffer at an appropriate temperature and reaction condition (e.g., 25° C. for 1 h). Annealing/hybridization reaction may be performed with detergent to mitigate aggregation of hydrophobic molecule 301. Any unbound molecules can be removed and/or washed via changing of the overlaid solution. If scaffold 201 is kept unaffixed in solution, centrifugation (e.g., ultracentrifugation) or alternative separation step can be performed to remove excess molecules.

As shown in FIG. 4B, a scaffold system comprises scaffold 221 as depicted in FIG. 2C. Accordingly, scaffold 221 includes a first end 223 and second end 225, each of which are open with a lumen 227 therebetween. Scaffold 221 further comprises second tether 229 extending toward lumen 227. Second tether 229 comprises a nucleic acid oligomer (or aptamers) configured to complement an oligomer (or aptamer) with attached molecule 303. Annealing/hybridization reaction of 229 with 303 may be performed with detergent to mitigate aggregation of hydrophobic molecule 303. Near second end 225 of scaffold 221 are a plurality of tethers 231, where one or more tethers of the plurality of tethers 231 are nucleic acid oligomer (or aptamer) configured to be connectors and complement a nucleic acid oligomer (or aptamer) connector of scaffold 201 or connect directly to scaffold 201. One or more of the plurality of tethers 231 can further provide a means for attaching a hydrophobic seed explained ahead.

The scaffolded liposome system is further synthesized by annealing/hybridizing tether 229 with an oligomer (or aptamer) with attached molecule 303. The annealing/hybridization reaction can be performed with a suitable buffer at an appropriate temperature and reaction condition (e.g., 25° C. for 1 h). The reaction can be performed with scaffold 221 affixed to a substrate or unaffixed in solution; similar to the case explained in FIG. 4A. If scaffold 221 is affixed to a substrate, any unbound molecules can be removed and/or washed via changing of the overlaid solution. Scaffold 221 can then be released from the substrate via strand displacement or cleavage of a labile chemical structure prior to the next step of conjoining with scaffold 201. If scaffold 221 is kept unaffixed in solution, centrifugation (e.g., ultracentrifugation) or alternative separation step can be performed to remove excess molecules.

Scaffold 221 can be brought into solution with scaffold 201, which is affixed to substrate 305 (FIG. 4C). The scaffolded liposome system is further synthesized by annealing/hybridizing one or more tethers of plurality of tethers 211 of scaffold 201 with one or more tethers of plurality of tethers 231 of scaffold 221 or by connecting one or more tethers of plurality of tethers 211 directly to scaffold 221 or by connecting one or more tethers of plurality of tethers 231 connecting directly to scaffold 201. This brings scaffold 201 to conjoin with scaffold 221. Specifically, second end 205 of scaffold 201 abuts second end 225 of scaffold 221 such that lumen 207 and lumen 227 join together to form one conjoined lumen. One or more tethers of plurality of tethers 211 or plurality of tethers 231 further complements with a plurality of oligomers (or aptamers) with attached hydrophobic seed 241. These annealing/hybridization reactions can be performed concurrently or subsequently in solution with a suitable buffer at an appropriate temperature and reaction condition (e.g., 25° C. for 1 h). Annealing/hybridization of oligomers with attached hydrophobic seed may be performed with detergent to mitigate aggregation. Any unbound scaffolds and/or hydrophobic seeds can be removed and/or washed via changing of the overlaid solution concurrently or in subsequent steps. If scaffold 201 is kept unaffixed in solution, centrifugation (e.g., ultracentrifugation) or alternative separation step can be performed to remove scaffolds that failed to conjoin. The product of annealing reactions is depicted in FIG. 4D.

With hydrophobic seeds 241 projecting towards the conjoined lumen upon completion of the annealing/hybridization reaction, a liposome can be formed. Lipid membrane components (e.g., but not limited to, any mixture of lipids, cholesterol) can be added to the solution. The lipid membrane components will associate with hydrophobic seeds 241 and form liposome 307 within the conjoined lumen (FIG. 4E). Liposome formation can be performed in solution at an appropriate temperature, suitable buffer with detergent, and reaction condition. In some implementations, detergent is slowly removed using buffer exchange technique (e.g., dialysis) to help formulate the liposomes. As liposome 307 forms, the hydrophobic portion of molecule 301 and of molecule 303 will integrate within the lipid bilayer of liposome while maintaining tethered to scaffold 201 and scaffold 221, respectively. Any free lipid membrane components remaining in solution can be removed and/or washed via changing of the overlaid solution. If scaffold 201 is kept unaffixed in solution, centrifugation (e.g., ultracentrifugation) or alternative separation step can be performed to remove excess lipid membrane components.

Upon liposome 307 formation and integration of molecule 301 and molecule 303 within the lipid bilayer of liposome, a reaction can be performed to release the molecules from their tether (FIG. 4F). In some implementations, strand displacement is performed to release molecule 301 and molecule 303 from scaffold 201 and scaffold 221, respectively. In some implementations, cleavage of a labile chemical structure is performed to release molecule 301 and molecule 303 from scaffold 201 and scaffold 221, respectively. With molecule 301 and molecule 303 released to move freely within the lipid bilayer of liposome, their kinetics, dynamics, and/or interaction can be assessed. In some implementations, a stimulus is added to the scaffolded liposome system to induce interaction between molecule 301 and molecule 303. In some implementations, scaffold 201 is detached from substrate 305 to perform the assessment, which can be performed by strand displacement or cleavage of a labile chemical structure of tether 213. In some implementations, molecule 301 and molecule 303 comprise a molecular tag to detect and/or monitor their behavior. In some implementations, molecule 301 and molecule 303 comprise one or more fluorophores, a donor-acceptor fluorophore pair, and/or a quencher to detect and/or monitor their behavior. In some implementations, a detector, a camera, or a plate reader instrument is utilized to detect and/or monitor the behavior of molecule 301 and molecule 303. In some implementations, single-molecule microscopy or total internal reflection fluorescence (TIRF) detection is utilized to detect and/or monitor the behavior of molecule 301 and molecule 303. In some embodiments, an optical system is utilized to impinge light energy onto a fluorophore of molecule 301 and/or molecule 303.

While specific examples and methods are depicted and described in reference to FIGS. 1A to 4F, it should be understood that these are examples and that various components and/or steps may be added, removed, and/or altered. Accordingly, the examples depicted are no meant to be construed as the entire scope of the various embodiments of the disclosure, but instead to be construed as one particular implementation.

1. A system for assessing lipid membrane-associated molecules, comprising:
a molecular scaffold having a tubular shape with open terminal ends;
one or more molecules, each molecule of the one or more molecules reversibly tethered to the molecular scaffold within a lumen of the molecular scaffold; and
a plurality of hydrophobic seeds tethered to the molecular scaffold within a lumen of the molecular scaffold.

2. The system of claim 1 further comprising two or more molecular scaffolds, wherein each molecular scaffold has a tubular shape and is conjoined with another molecular scaffold in an end-to-end manner such that the lumen of each molecular scaffold is conjoined.

3. The system of claim 2, wherein each molecular scaffold comprises one or more molecules reversibly tethered thereto within its lumen.

4. The system of claim 2, wherein each molecular scaffold is conjoined with another molecular in an end-to-end manner such that gaps between the conjoined molecular scaffolds are mitigated or absent.

5. The system of claim 2, wherein each molecular scaffold is conjoined with another molecular via complementary oligomeric nucleic acids.

6. The system of claim 2, wherein each molecular scaffold is conjoined with another molecular such that the plurality of hydrophobic seeds tethered to the molecular scaffold are patterned in a manner such that it can allow a molecule to pass thereby via a lipid membrane of a liposome formed by the plurality of hydrophobic seeds.

7. The system of claim 1, wherein the molecular scaffold comprises DNA origami.

8. The system of claim 1, wherein the molecular scaffold is configured and the one or more molecules are tethered to the scaffold in a manner such that the one or more molecules are sterically hindered from interacting with a molecule tether within a lumen of another molecular scaffold.

9. The system of claim 1, wherein each molecule of the one or more molecules is capable of associating with a lipid membrane.

10. The system of claim 9, wherein at least one molecule of the one or more molecules comprises a hydrophobic domain.

11. The system of claim 9, wherein at least one molecule of the one or more molecules comprises an attached hydrophobic moiety.

12. The system of claim 9, wherein at least one molecule of the one or more molecules comprises a transmembrane protein, a peripheral membrane protein, a nucleic acid with attached hydrophobic domain, a lipid, a fatty acid, a phospholipid, a glycolipid, a sphingolipid, a glycerolipid, a sterol, cholesterol, a prenol, a saccharolipid, a tocopherol, a hydrocarbon chain, and a polyketide.

13. The system of claim 1, wherein the reversible tether a nucleic acid oligomer.

14. The system of claim 13, wherein strand displacement is capable of untethering the molecule.

15. The system of claim 1, wherein the reversible tether comprises a labile chemical structure.

16. The system of claim 15, wherein the tether is capable of being cleaved via energy to break the labile chemical structure.

17. The system of claim 1, wherein the plurality of hydrophobic seeds tethered to the molecular scaffold are patterned in a manner such that it can allow a molecule to pass thereby via a lipid membrane of a liposome formed by the plurality of hydrophobic seeds.

18. The system of claim 1 further comprising a liposome formed within the lumen of the scaffold.

19. The system of claim 18, wherein the liposome is formed via the plurality of hydrophobic seeds and lipid membrane components.

20. The system of claim 18, wherein the one or more molecules are integrated within a lipid membrane of the liposome.

21.-40. (canceled)
